(19) United States
(12) Patent Application Publication
(10) Pub. No.: US 2023/0183642 A1
(43) Pub. Date: Jun. 15, 2023

(54) METHOD OF PRODUCING PLANT-DERIVED EXOSOMES

(71) Applicant: YEDITEPE UNIVERSITESI, Istanbul (TR)

(72) Inventors: Fikrettin SAHIN, Istanbul (TR); Bahar SOGUTMAZ OZDEMIR, Istanbul (TR); Batuhan Turhan BOZKURT, Istanbul (TR); Oguz Kaan KIRBAS, Istanbul (TR); Pakize Neslihan TASLI, Istanbul (TR); Umit Cem DERMAN, Istanbul (TR)

(73) Assignee: YEDITEPE UNIVERSITESI, Istanbul (TR)

(21) Appl. No.: 17/924,699

(22) PCT Filed: May 10, 2021
(86) PCT No.: PCT/TR2021/050454

§ 371 Date: Nov. 11, 2022

(30) Foreign Application Priority Data
May 11, 2020 (TR) 2020/07334

(51) Int. Cl. C12N 5/04

(52) U.S. Cl. CPC C12N 5/04

(57) ABSTRACT

A method of producing plant-derived exosomes from plant tissue culture based cell suspension cultures is provided. The method includes: obtaining the plant tissue culture based cell suspension culture; mixing the the plant tissue culture based cell suspension culture with an isolation solution; centrifuging to obtain a supernatant and an infranatant; and obtaining the plant-derived exosomes from the infranatant. The objective of the present invention is to produce homogenous plant exosomes with high volume and purity by making use of the advantages of the plant suspension culture to be used for purposes such as therapeutics and drug carriers.

### CROSS REFERENCE TO THE RELATED APPLICATIONS

This application is the national phase entry of International Application No. PCT/TR2021/050454, filed on May 10, 2021, which is based upon and claims priority to Turkish Patent Application No. 2020/07334, filed on May 11, 2020, the entire contents of which are incorporated herein by reference.

### TECHNICAL FIELD

The present invention relates to a method of obtaining plant-derived exosomes from plant tissue culture-based cell suspension cultures.

### BACKGROUND

In transport and storage of substances within the cell, small sacs which are called vesicles and are separated by a lipid bilayer from the cytoplasm fluid, are involved. Exosomes are vesicles, which are released by many organisms from prokaryotes to high eukaryotes including the plants, and which contain lipid bilayer vesicles of different sizes [1]. The said vesicles have the capacity^{,} of transferring information to the other cells in order to influence the cell function. Signal transfer via exosomes is carried out by means of biomolecules in many different categories consisting of proteins, lipids, nucleic acid and sugars. Since their discovery, many different applications of exosomes have been developed in the fields of biology and medicine. For example, the use of exosomes in the pathogenesis, diagnosis and treatment of cancer, immune system diseases, and neurodegenerative diseases such as ALS and Alzheimer's is known. In addition to these, there are many studies on the use of exosomes as carriers in drugs and gene therapy methods such as CRISPR-Cas9 due to the fact they are cell-driven and have the ability to cross the blood-brain barrier [2].

In the state of the art, exosome studies are mainly carried out with human exosomes. With exosomes obtained from different cell lines, body fluids and individuals and cell lines showing different diseases such as cancer, almost an entire exosome map of the humans has been drawn. All eukaryotic creatures, including plants, produce exosomes. The limited number of studies conducted to date on exosomes produced by plants has shown that grapefruit [3] and lemon (Citrus lemon) [4] produce exosotnes, and that these exosomes suppress the growth of cancer cells by in vitro and in vivo studies. Another study in the field of the invention demonstrated the interactions of the exosomes obtained from four different plants with mouse cells, proving that plant exosomes affect mammalian cells by crossing the species barrier [5]. There are also few studies on the effects of plant exosomes on the plant itself. There are studies showing that a plant secretes exosomes to protect itself under pathogen stress [6].

Plant exosome studies pose several challenges compared to human exosome studies. This is one of the most important reasons for the limited number of plant exosome studies. Plants used in exosome studies are obtained from regional markets. However, plants grown under uncontrolled conditions and kept waiting for a long time after harvest may cause unexpected results between the trials. In addition, when extracting exosomes from immature tissues such as fruits, there are many phytochemicals (different bioactive substances produced by plants) that need to be removed. If these difficulties are overcome, plant-derived exosomes have the potential to show the effects that they show on cancer in the treatment of other diseases as well [3, 4]

Plants have followed an evolutionary process in which they developed defense mechanisms that could protect themselves against situations where they could be harmed in their habitats due to their inability to move [7]. Therefore, it is possible for them to develop different molecular pathways and produce a large number of special molecules. These molecules have been used for a long time in many industries such as medicine, food, paint, cosmetics and the like [8]

Although many plant-based molecules or products have been found, this field is still open to new studies and discoveries [9]. For many years, all plants that have completed their development are used for the production of plant-based molecules. However, it has recently been demonstrated that plant cell suspension cultures are more suitable for producing plant-derived products. Plant cell suspension cultures are performed by regularly mixing callus cultures in liquid medium and keeping variables such as light, humidity and temperature constant [10]. Compared to all plants that have completed their development, plant cell suspension cultures enable to achieve higher mass yield in a shorter period of time [9]. In addition, plants grown in the soil have the potential to carry a risk of contamination such as biological pathogens or pesticide residues [11]. Soil-based agriculture has uncontrolled environmental conditions compared to cell suspension cultures [12].

In addition to having a stable yield, plant cell suspension cultures allow obtaining stable and reproducible plant-based products. The fact that environmental factors are constant prevents products from being affected by ordinary changes. The fact that the produced cells are single cell clones also ensures consistency. In addition, the use of plant cell suspension culture makes post-production processes easier. A simple filtration or centrifuge process separates the plant cells and the suspension medium from each other. Other advantages thereof include the large scale production of cell suspension cultures and the potential for scaling up [13].

In the recent years, studies on exploring the activities of plant-derived exosomes in biological systems and particularly on analyzing their molecular contents and n.anovesicular structures have gained momentum. Consequently, very high amounts of plant-derived exosomes with very high purity are required to be used in studies. As a result of the fact that today's exosome isolation methods are developed to be cell targeted, the solutions obtained by the use of whole plants or their fruits contain too much contamination to be isolated by these methods. For this reason, the purity of the exosomes, which are obtained from fruits from local markets or samples studied as whole plants, causes considerable doubt with respect to the studies.

In the studies conducted on exosomes obtained from mammalian cell cultures, it has been determined that the cells regulate and change the molecular contents of the exosomes that they secret depending on the ambient conditions. Accordingly, it is expected that the biomolecular contents of the exosomes secreted by the plant cells will also be highly affected by the ambient conditions. For the characteristics of a plant in nature, in addition to the natural factors such as the salinity of the soil, the availability of important minerals and trace elements in the soil, the amount of moisture in the air and the amount of light in the environment; artificial factors and occasional natural events are of high importance as weft As a result of these, it is very difficult to obtain a single standard exosome, which has the same characteristics homogeneously and whose properties have not changed over time and are thought not to change in the future, from plants that continue to grow in the abovementioned manner (in uncontrolled environments). For this reason, reproducible results shown in sub-studies to be continued following the experimental process of the scientific studies in the literature are approached with suspicion. In addition to these, in fruit-based studies used to obtain exosornes, there are no significant attempts to conduct studies which are both uninterrupted in terms of time and large-scale due to the fact that they are dependent on the plant's crop production schedule.

The European patent document no. EP3576844, an application known in the art, relates to a plant-derived exosome for use in cancer treatment and wound healing. The invention disclosed in the said document enables to provide a low-cost product, which does not cause toxic effects in human body, does not cause damage in the healthy cells during the course of the cancer treatment, and does not pose any infection risk. In the product of the invention, wheatgrass, garlic and ginger can be used alone or in combination in the invention as the plant source.

The United States patent application document no. US2018271773 A1 relates to a composition containing extracellular vesicles produced from plant juice. The said extracellular vesicles have excellent skin condition-improving effects such as skin whitening, moisturizing and wrinkle reducing effects, and exhibit an effect of preventing hair loss.

### SUMMARY

The objective of the present invention is to produce plant exosomes with high volume and purity by making use of the advantages of the plant suspension culture to be used for purposes such as therapeutics and drug carriers.

Another objective of the present invention is to provide a homogenous exosome culture.

### BRIEF DESCRIPTION OF THE DRAWINGS

Figures of the method of producing plant-derived exosomes of the present invention are described as follows:

FIG. 1 shows the microscopic images of the cultured tobacco cells.

FIG. 2 shows the microscopic images of the cultured stevia cells.

FIG. 3 shows the measurement of the distribution of the sizes of the exosomes obtained from stevia plant cell suspension culture via dynamic light scattering.

FIG. 4 shows the measurement of the distribution of the sizes of the exosomes obtained from stevia plant cell suspension culture via dynamic light scattering.

FIG. 5 shows the view of the morphologies and sizes of the exosomes obtained from tobacco plant from different plant cultures by SEM image.

FIG. 6 shows the view of the morphologies and sizes of the exosomes obtained from stevia plant from different plant cultures by SEM image.

FIG. 7 shows a graphical representation of the control group carried out for characterization of the exosomes obtained from tobacco cells by flow cytometry.

FIG. 8 shows a graphical representation of the measurement of the CD 9 proteins of the exosomes obtained from tobacco cells by flow cytometry.

FIG. 9 shows a graphical representation of the measurement of the CD 63 proteins of the exosomes obtained from tobacco cells by flow cytometry.

FIG. 10 shows a graphical representation of the measurement of the HSP70 proteins of the exosomes obtained from tobacco cells by flow cytometry.

FIG. 11 shows a graphical representation of the control group carried out for characterization of the exosomes obtained from stevia cells by flow cytometry.

FIG. 12 shows a graphical representation of the measurement of the CD9 proteins of the exosomes obtained from stevia cells by flow cytometry.

FIG. 13 shows a graphical representation of the measurement of the CD63 proteins of the exosomes obtained from stevia cells by flow cytometry.

FIG. 14 shows a graphical representation of the measurement of the IHSP70 proteins of the exosomes obtained from stevia cells by flow cytometry.

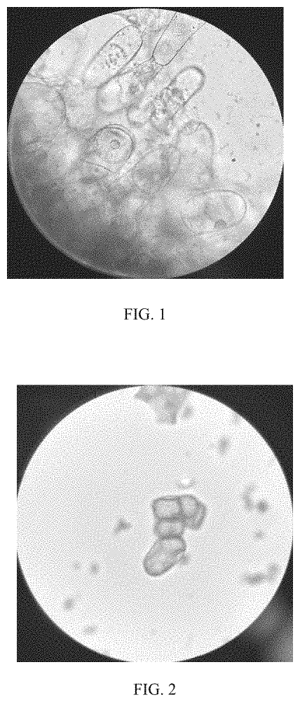

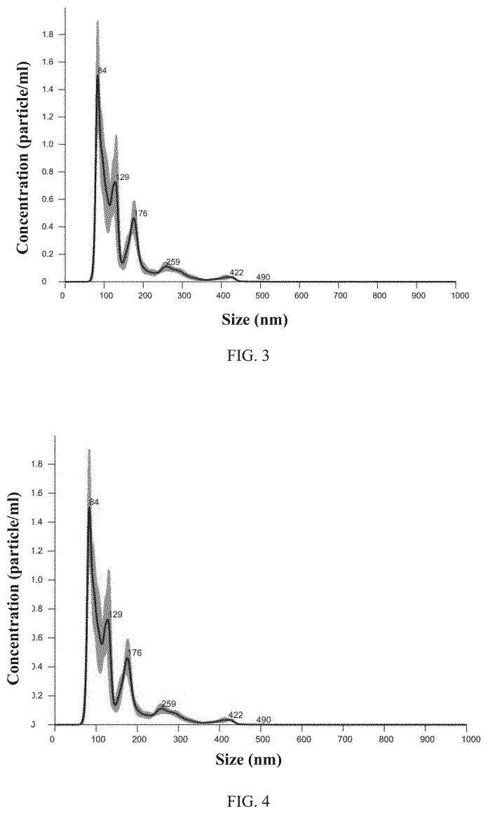

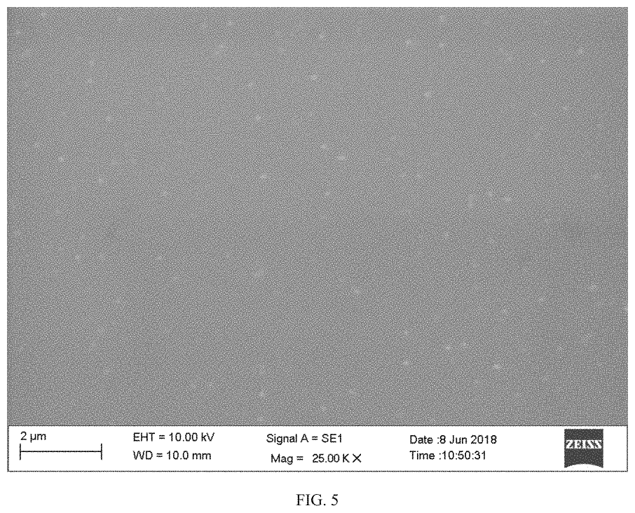

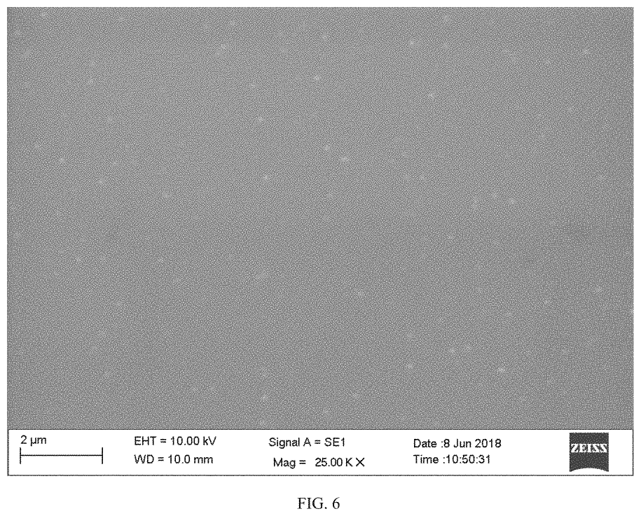

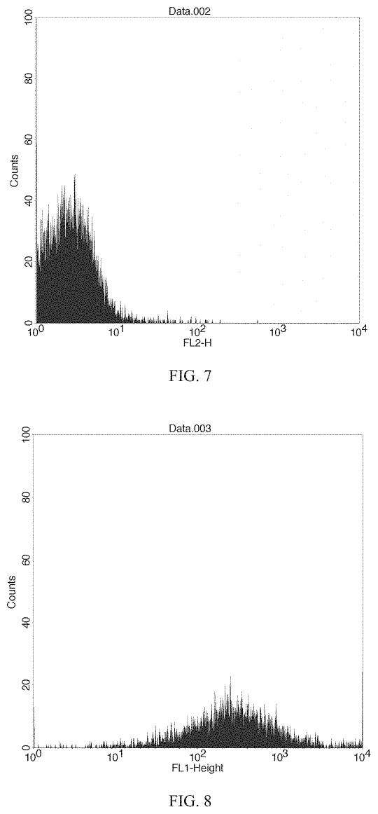

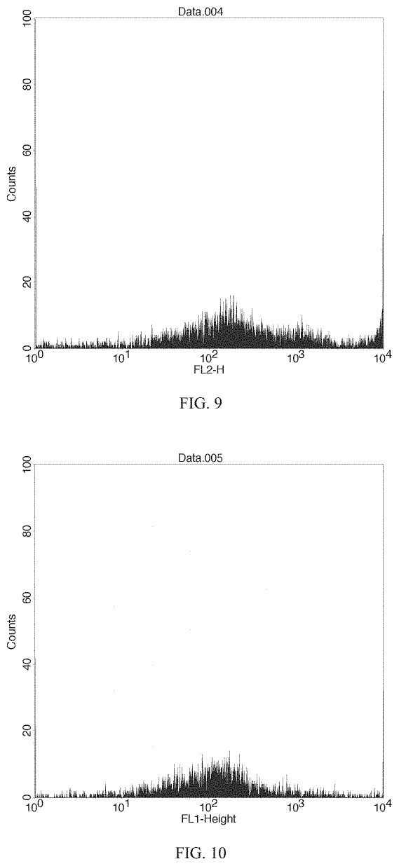

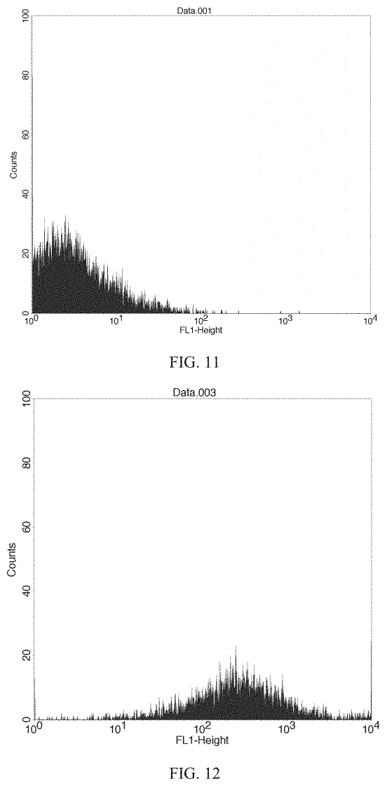

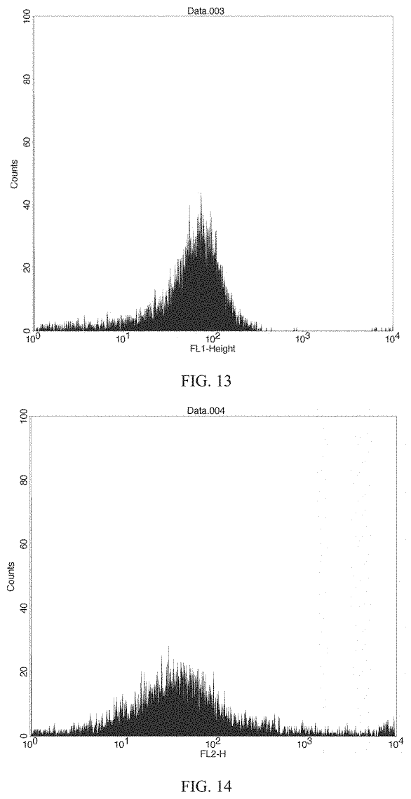

### DETAILED DESCRIPTION OF THE EMBODIMENTS

The present invention relates to a method of producing plant-derived exosomes from plant tissue culture-based cell suspension cultures comprising the following steps:
Obtaining the plant cell suspension culture,
   a Making the regularly subcultured callus culture, whith is obtained from plants (preferably from tobacco leaves or stevia leaves) by wounding method, ready to be transferred to liquid culture within 2-3 weeks after subculturing,
   Dividing the callus culture into small pieces of 1-5 mm and placing the pieces into Erlenmeyer flasks such that the flasks will be 10-50% full,
   Preparing the liquid culture medium in the Erlenmeyer flask such that it will contain 20-30 g/L sucrose, (in case tobacco leaves are used 0.1-0.8 mg/L or in case stevia leaves are used 1-4 mg/L) 6-Benzylaminopurine, 1-3 mg/L 1-Napthaleneacetic acid, 3.5-4.5 g/L Murashige & Skoog vitamin-containing salt mixture,
   Maintaining the liquid culture continuously under light during growth and agitating at an agitation speed of 80-120 rpm at a temperature of 20-26° C.,
   Performing sub-culture via vacuum filtration system at intervals of 5-10 days,
   Straining through a sterile steel sieve once every 3-5 subcultures,
Mixing the plant culture media with the isolation solution containing 2-1% polyethylene glycol with a molecular weight of 25-45 kDa and 1-2% dextran with a molecular weight of 450-650 kDa at a ratio of 1:1 by inverting 20 times,
Centrifuging at 1500 g for 10 minutes at +4° C.,
After the centrifugation process, obtaining two separated phases as the supernatant comprising 90% of the total and containing protein and other cellular wastes, and the infranatant comprising 10% and where exosomes are collected,
Carefully pulling and discarding the supernatant,
Transferring the infranatant containing exosomes to a clean tube,
Obtaining Solution C as the supernatant of the aqueous two-phase system obtained by diluting the isolation solution with water at a ratio of 1:1 and centrifuging at 1000 x g for 10 minutes,
Adding solution C at a ratio of 1:1 to the said infranatant containing exosome and inverting it 10 times,
Centrifuging the mixture at 12000-14000 g for 10 minutes at +4° C.,
Upon collection of the supernatant, removing the ethanol (EtOH) in solution C by means of an evaporator,
Storing the obtained exosomes as the final product (at −80° C. for up to 12 months upon aliquoting, or at +4° C. for up to 36 months in powder form upon lyophilizing).

The present invention relates to a method of producing plant-derived exosomes from plant tissue culture-based cell suspension cultures. In the said method, firstly, tobacco and stevia cell suspension cultures are created, then plant exosomes are obtained by using the said cell suspension cultures.

Within the scope of the invention, the culture medium is ensured to be treated with sugar, salt, vitamins and hormones. In this process, 6-Benzylaminopurine is preferred as the hormone (6-Benzylaminopurine; benzyl adenine, BAP or BA is a first-generation synthetic cytokinin that promotes plant growth and development responses, setting blossoms and stimulating fruit richness by stimulating cell division. Callus tissue, which is regularly subcultured and obtained from plants (preferably tobacco leaves or stevia leaves) by wounding method, is formed by stimulating the said leaf tissues with suitable hormone concentrations. Callus culture is prepared in which the properties of the obtained callus tissue are continuously preserved with the help of certain hormones. Tobacco and Stevia are different plant species, and they need to be regularly stimulated with certain hormones to protect the callus culture. These hormones vary between species. Sugar, salt and vitamins can also vary, but the same sugar and salt ratios have been found to be suitable for Tobacco and Stevia. These hormones vary between species. Sugar, salt and vitamins can also vary. Murashige & Skoog salt mixture containing vitamins [14], which is widely used in the state of the art, is considered as the vitamin-salt mixture used herein. Said Murashige & Skoog mixture, named after the researchers who invented it, is a medium composition frequently used in plant tissue culture. The “vitamin salt mixture” referred to herein is a liquid-liquid medium obtained using Murashige & Skoog powder. Amount of Murashige & Skoog used is prepared such that “Murashige & Skoog vitamin-containing salt mixture comprises [10] 3,5-4,5 g/L [11].

It has been determined in the studies conducted in present time that by isolating the exosomes obtained from plants from the medium used in plant cell culture, very important advantages have emerged in terms of the homogeneity of the exosomes, amount of production and genetic applications. The problem of not being able to obtain a homogeneous exosome culture, which is one of the most important obstacles in studying the bioactivity of plant-derived exosomes, has been solved within the scope of the invention. In the method of the invention, exosomes are secreted into the medium in the plant cell suspension culture by a single cell type and the said cells are grown under controlled conditions. Thus, it is possible over time to minimize the vesicular structure and content differences that will occur during the production of exosomes required in experimental studies carried out. Another advantage of this is that the medium used in plant tissue culture contains much less contamination than a fruit extract to be used for exosome purification. Accordingly, significant advantages are obtained in exosome isolation in terms of both time and efficiency.

Within the scope of the invention, a process is provided for the purification of exosomes from plants, which is generally independent from the growth conditions and areas required for plants. In the field of the invention, in fruit-based studies, the problems such as the facts that the plants have specific dates when they characteristically yield products and that the amount of area required to obtain the desired amount of exosomes is too large are overcome with the method of the present invention. By using bioreactors for plant cultures, the exosomes of the cells intended to be studied in plants can be obtained independent from time and in very high amounts in more minimal areas.

Within the scope of the present invention, obtaining exosomes from the cell population in plant tissue culture enables to examine the responses of plant cells to environmental changes. Moreover, by incorporating a special protein into the vesicular structure in the regulation of plant-derived exosome cargoes, it will be possible to make the responses of genetic changes on plant cells cell-specific.

Within the scope of the invention, aliquoting or lyophilization processes are applied to preserve the exosome, which is the final product, for a long time. Aliquoting is for preventing exposure to a repetitive freeze-thaw process. Lyophilization provides a long-term stability at +4 degrees. These processes are used for the correct storage of our final product.

### REFERENCES

[1]. Thery, C., Zitvogel, L., & Amigorena, S. (2002) Exosomes: composition, biogenesis and function. Nature Reviews Immunology, 2(8), 569.
[2]. Corrado, C, Raimondo, S., Chiesi, A., Ciccia, F., De Leo, G., & Alessandro, R. (2013). Exosomes as intercellular signaling organelles involved in health and disease, basic science arid clinical applications. International journal of molecular sciences, 14(3), 5338-5366.
[3]. Zhuang, X., Teng, Y., Samykutty, A., Mu, J., Deng, Z., Zhang, L., . . . & Zhang, H. G. (2016). Grapefruit-derived nanovectors delivering therapeutic miR17 through an intranasal route inhibit brain tumor progression. Molecular Therapy, 24(1), 96-105.
[4]. Raimondo, S., Naselli, F., Fontana, S., Monteleone, F., Dico, A. L., Saieva, L., . . . & De Leo, G. (2015). Citrus limon-derived nanovesicles inhibit cancer cell proliferation and suppress CML xenograft growth by inducing TRAIL-mediated cell death. Oncotarget, 6(23), 19514.
[5]. Ju, S., Mu, J., Dokland, T., Zhuang, X., Wang, Q., Jiang, H., . . . & Roth, M (2013). Grape exosome-like nanoparticles induce intestinal stem ceils and protect mice from DSS-induced colitis. Molecular Therapy, 21(7), 1345-1357.
[6]. Meyer, D., Pajonk, S., Micali, C., O'Connell, R., & Schulze-Lefert, P. (2009). Extracellular transport and integration of plant secretory proteins into pathogen-induced cell wall compartments The Plant Journal, 57(6), 986-999.
[7]. Z̆ádniková, P., Smet, D, Zhu, Q., Straeten, D. V. D., & Benková, E. (2015). Strategies of seedlings to overcome their sessile nature auxin in mobility control. Frontiers in Plant Science, 6, 218.
[8]. Fabricant, D. S., & Farnsworth, N. R. (2001). The value of plants used in traditional medicine for drug discovery. Environmental health perspectives, 109(suppl 1), 69-75.
[9]. Rao, S. R., & Ravishankar, G. A. (2002). Plant cellcultures- chemical factories of secondary metabolites. Biotechnology advances, 20(2), 101-153.
[10]. Mustafa, N. R., De Winter, W., Van Iren, F, & Verpoorte, R. (2011) Initiation, growth and cryopreservation of plant cell suspension cultures. Nature protocols, 6(6), 715.
[11]. Hellwig, S., Drossard, J., Twyman, R. M., & Fischer, R. (2004) Plant cell cultures for the production of reconibinant proteins. Nature biotechnology, 22(11), 1415.
[12]. Nalawade, S. M., & Tsay, H. S. (2004). In vitro propagation of some important Chinese medicinal plants and their sustainable usage. In Vitro Cellular & Developmental Biology—Plant, 40(2), 143-154.
[13]. Yue, W., Ming, Q. L., Lin. B, Rahman, K. Zheng, C. J., Han, T., & Qin, L. P. (2016) Medicinal plant cell suspension cultures: pharmaceutical applications and high-yielding strategies for the desired secondary metabolites. Critical reviews in biotechnology, 36(2), 215-232.
[14]. Murashige, T., & Skoog, F. (1962) A revised medium for rapid growth and bioassays with tobacco tissue cultures. Physiologiaplantarum, 15(3), 473-497.

What is claimed is:

1. A method of producing plant-derived exosomes from a plant tissue culture based cell suspension culture comprising the following steps:
obtaining the plant tissue culture based cell suspension culture,
making a regularly subcultured callus culture from plants by a wounding method, ready to be transferred to a liquid culture within 2-3 weeks after a subculturing,
dividing the regularly subcultured callus culture into pieces of 1-5 mm and placing the pieces into Erlenmeyer flasks such that the Erlenmeyer flasks are 10-50% full,
preparing a liquid culture medium in each of the Erlenmeyer flasks such that the liquid culture medium contains sucrose, 6-Benzylaminopurine, 1-Napthaleneacetic acid, and a Murashige & Skoog vitamin-containing salt mixture,
maintaining the liquid culture medium continuously under light during growth and agitating the liquid culture medium at an agitation speed of 80-120 rpm at a temperature of 20-26° C.,
performing a sub-culture via a vacuum filtration system at intervals of 5-10 days,
straining through a sterile steel sieve once every 3-5 subcultures,
mixing the plant tissue culture based cell suspension culture with an isolation solution at a ratio of 1:1 by inverting 20 times to obtain a first resulting mixture,
centrifuging the first resulting mixture at 1500 g for 10 minutes at +4° C.,
after a centrifugation process, obtaining two separated phases as a supernatant accounting for 90% and comrpising a protein and other cellular wastes and an infranatant accounting for 10% and comprising the plant-derived exosomes,
pulling and discarding the supernatant,
transferring the infranatant containing the plant-derived exosomes to a clean tube,
obtaining a solution as a supernatant of an aqueous two-phase system obtained by diluting the isolation solution with water at a ratio of 1:1 and centrifuging at 1000×g for 10 minutes,
adding the solution at the ratio of 1:1 to the infranatant containing the plant-derived exosomes to obtain a second resulting mixture and inverting the second resulting mixture 10 times,
centrifuging the second resulting mixture at 12000-14000 g for 10 minutes at +4° C.,
upon a collection of a supernatant from the second resulting mixture, removing ethanol (EtOH) in the solution by an evaporator to obtian the plant-derived exosomes,
storing the plant-derived exosomes as a final product.

2. The method of producing the plant-derived exosomes according to claim 1, wherein a plant tissue used is tobacco leaves.

3. The method of producing the plant-derived exosomes according to claim 1, wherein a plant tissue used is stevia leaves.

4. The method of producing the plant-derived exosomes according to claim 1, wherein the liquid culture medium in each of the Erlenmeyer flasks is prepared such that it will the liquid culture medium contains 20-30 g/L of the sucrose, 6-Benzylaminopurine, 1-3 mg/L, of the 1-Napthaleneacetic acid, 3.5-4.5 g/L of the Murashige & Skoog vitamin-containing salt mixture.

5. The method of producing the plant-derived exosomes according to claim 1, wherein the liquid culture medium in each of the Erlenmeyer flasks comprises 0.1-0.8 mg/L of the 6-Benzylaminopurine when tobacco leaves are used as a plant tissue.

6. The method of producing the plant-derived exosomes according to claim 1, wherein the liquid culture medium in each of the Erlenmeyer flasks comprises 1-4 mg/L of the 6-Benzylaminopurine when stevia leaves are used as a plant tissue.

7. The method of producing the plant-derived exosotnes according to claim 1, wherein the plant-derived exosotnes are the final product and aliquoted and stored at −80° C. for up to 12 months.

8. The method of producing the plant-derived exosomes according to claim 1, wherein the plant-derived exosomes are the final product and lyophilized and stored at +4° C. for up to 36 months in a powder form.
